(19) United States
(12) Patent Application Publication
(10) Pub. No.: US 2026/0085163 A1
(43) Pub. Date: Mar. 26, 2026

(54) ENZYME-CONTAINING SINGLE-LAYER ARTICLE HAVING WATER BARRIER PROPERTIES

(71) Applicant: CARBIOLICE, Clermont-Ferrand (FR)

(72) Inventor: Vincent LEGRAND, Combronde (FR)

(21) Appl. No.: 19/108,786

(22) PCT Filed: Sep. 14, 2023
(86) PCT No.: PCT/EP2023/075346

§ 371 Date: Mar. 5, 2025

(30) Foreign Application Priority Data
Sep. 14, 2022 (FR) FR2209252

(51) Int. Cl. C08J 5/18; C08L 1/02; C08L 67/03; C08L 67/04; C12N 9/54

(52) U.S. Cl. CPC C08J 5/18; C08L 1/02; C08L 67/03; C08L 67/04; C12N 9/54; C08J 2301/02; C08J 2367/03; C08J 2367/04; C08L 2201/06; C12Y 304/21014

(57) ABSTRACT

The present invention relates to an enzyme-containing biodegradable single-layer article having water barrier properties.

### FIELD OF THE INVENTION

The present invention relates to a biodegradable single-layer plastic article comprising a mixture of polymers, including a biodegradable polyester and enzymes capable of breaking it down.

### PRIOR ART

Bio-based and biodegradable articles, in particular single-layer or multi-layer articles, are well-known for use in the bag industry, and in particular for manufacturing plastic bags. These bags are in particular used for packaging food products, and in particular fruit and vegetables.

Reference is made in particular to the articles described in the patents and patent applications WO 2020/193770, WO 2007/118828, WO 2002/059202A1, WO 2002/059199, WO 2002/059198, WO 2004/052646, WO 2018/233888, WO 2021/005205, U.S. Pat. Nos. 6,841,597, 8,751,816, 5,436,078, 9,096,758, US 2009/324917 and CN 106881929. However, these articles do not have water barrier properties for food packaging applications.

The process for producing an article comprising enzymes capable of breaking down the polymers of which it is composed must both preserve the enzymes during the preparation of said article, for example via mixtures with polymers which melt at temperatures which do not degrade said enzymes, and satisfy the physical property requirements.

The simple and cost-effective improvement of the water barrier property of an enzyme-containing biodegradable article is all the more difficult to achieve given that this must not be at the expense of the activity of the enzymes in breaking down the biodegradable polyester.

It is therefore necessary to propose solutions for improving the water barrier properties of a plastic article without impairing its biodegradability in a simple, cost-effective and efficient manner.

This objective, inter alia, is achieved by the new composition of the single-layer article according to the invention, which provides a number of advantages, in particular its water barrier properties.

In fact, the inventors discovered that a specific mixture of polymers improves the water barrier properties of a biodegradable plastic single-layer article comprising enzymes and satisfies the physical property requirements of the articles made with this mixture.

### SUMMARY OF THE INVENTION

The invention relates to a biodegradable single-layer article, the composition of which comprises:
a) from 3% to less than 60% PHA (polyhydroxyalkanoate)
b) a biodegradable polyester different from PHA (a),
c) at least 0.001% enzymes capable of breaking down said degradable polyester (b), and
d) optionally, at least one complementary thermoplastic polymer, different from PHA (a) and the biodegradable polyester (b),
   the percentages being given in weight relative to the total weight of the composition of the article.

The article according to the invention relates to a flexible plastic article, in particular a plastic film, or a rigid plastic article, in particular a sheet.

The flexible article according to the invention preferably has a thickness ranging from 20 to 100 μm, preferably from 25 to 80 μm, more preferably from 30 to 60 μm, more preferably still of around 50 μm.

The rigid article according to the invention has a thickness comprised between 150 and 5000 μm, preferably comprised between 400 and 500 μm, more preferably equal to around 450 μm.

The invention also relates to a film comprising a single-layer article according to the invention, preferably a flexible article according to the invention.

The invention also relates to a sheet comprising a single-layer article according to the invention, preferably a rigid article according to the invention.

The article according to the invention may be used for preparing a package, seal, cup, plate, beverage capsule, tub or blister pack.

### DETAILED DESCRIPTION

### Definitions

The present description will be better understood with reference to the following definitions.

“Plastic article” according to the invention means any article made from at least one polymer, such as a sheet, film, tube, rod, profile, shape, solid block, fibre, etc. of plastic.

The term “plastic film” refers to a flexible plastic film (i.e. able to be folded without breaking) less than 250 μm thick. Thin films are considered to be less than 100 μm thick and are preferably produced by blown film extrusion, whereas thick films are more than 100 μm thick and are preferably produced by cast film extrusion.

According to the invention, the term “rigid article” or “plastic rigid article” refers to a plastic article which is not a film. These articles are preferably made by calendering, injection-moulding, thermoforming, blow-moulding or inflation or by rotational moulding and 3D printing.

As used here, the term “masterbatch” means a concentrated mixture of selected ingredients (such as enzymes, additives, etc.) and a carrier polymer with can be used to incorporate said ingredients into plastic articles in order to give them the desired properties. The masterbatch compositions enable the method to cost-effectively introduce selected ingredients during the plastic manufacturing process. The masterbatch is advantageously composed of a carrier polymer in which the selected ingredients are incorporated in high concentrations. Generally speaking, the masterbatch is intended to be mixed with one or more polymers or a polymer-based matrix in order to produce a final plastic article containing a desired quantity of selected ingredients. The masterbatch may also comprise mineral or organic fillers.

Unless otherwise stated, the percentages and relative ratios are expressed by mass in relation to the total mass of the composition of the article.

It should be noted that the expressions “from . . . to . . . ” and “comprised between . . . and . . . ” used in this description should be understood as including the limits mentioned. Conversely, the expressions “greater than . . . ”, “less than . . . ” and “more than . . . ” used in the present description do not include the limits mentioned.

In the context of the invention, the term “around” refers to a margin of +/−5%, preferably +/−1%, or within the tolerance of an appropriate measuring device or instrument.

### Composition of the Articles

The article according to the invention is a biodegradable plastic single-layer article, the composition of which comprises:
a) from 3% to less than 60% PHA (polyhydroxyalkanoate)
b) a biodegradable polyester different from PHA (a),
c) at least 0.001% enzymes capable of breaking down said biodegradable polyester (b), and
d) optionally at least one complementary thermoplastic polymer, different from PHA (a) and the biodegradable polyester (b), the percentages being given in weight relative to the total weight of the composition of the article.

Without being bound by any particular theory, it appears that the PHA (a), present in the composition in a quantity ranging from 3% to less than 60% by weight relative to the total weight of the composition of the article with a biodegradable polyester (a), and possibly at least one complementary thermoplastic polymer (d), exerts an enhanced water barrier function in an enzyme-containing single-layer article.

According to the invention, the PHA (polyhydroxyalkanoate) polymers are bidoegradable polymers and many of them are produced on an industrial scale by bacterial fermentation processes or isolated from plant matter such as maize, sweet potatoes, etc. The PHA polymers may also be polycondensates of one or more hydroxyalkanoic acids. Examples of such hydroxyalkanoic acids that can be comprised in the PHA polymer are glycolic acid, hydroxypropanoic acid (also known as lactic acid), hydroxy butyric acid, hydroxyisobutanoic acid, hydroxypentanoic acid (also known as hydroxyvaleric acid), hydroxyhexanoic acid (also known as polycaprolactone, PCL), hydroxyheptanoic acid, hydroxyoctanoic acid, hydroxydecanoic acid, hydroxydodecanoic acid, hydroxytetradecanoic acid, or combinations of two or more of these.

Examples include PHAs such as PHBV (poly-3-hydroxy butyrate-co-3-hydroxyvalerate), PHBH (poly-3-hydroxy butyrate-co-3-hydroxyhexanoate), PHB (poly-β-hydroxy butyrate), PHH (Polyhydroxyhexanoate), P3HP (poly 3-hydroxypropionate), P3HB (poly 3-hydroxy butyrate), P4HB (poly 4-hydroxy butyrate), P4HV (poly 4-hydroxyvalerate), P5HV (poly 5-hydroxyvalerate), PHB3HP (poly 3-hydroxy butyrate-co-3-hydroxypropionate), P3HB4HB (poly 3-hydroxy butyrate-co-4-hydroxy butyrate), PHB4HV (poly 3-hydroxy butyrate-co-4-hydroxyvalerate), PHB3HV (poly 3-hydroxy butyrate-co-3-hydroxyvalerate), PHB3HH (poly 3-hydroxy butyrate-co-3-hydroxyhexanoate), PHB5HV (poly 3-hydroxy butyrate-co-5-hydroxyvalerate), and mixtures thereof in any proportions. The PHA is preferably selected from PHBV or PHBH.

The PHA (a) content is preferably comprised between 5% and 50%, between 7% and 45%, between 10% and 40%, preferably between 10% and 30%, more preferably still between 12% and 27% by weight relative to the total weight of the composition of the article. According to some embodiments of the invention, the PHA content is equal to around 10%, 15%, 20%, 25%, 30%, 35%, 40%, 45%, 50% and 55% by weight relative to the total weight of the composition of the article.

The composition of the single-layer article according to the invention comprises a biodegradable polyester (b), different from PHA (a), and at least 0.001% enzymes capable of breaking down the biodegradable polyester (b) of the article.

Within the scope of the invention, “biodegradable polyester” means a polyester that can be broken down by enzymes, and more specifically enzymes used in the composition of said biodegradable single-layer article. The biodegradable polyester present in the single-layer article according to the invention is different from PHA. In other words, the single-layer article comprises PHA (a) and another biodegradable polyester (b) distinct from the PHA. Examples of biodegradable polyesters (b), different from PHA, include PCL (polycaprolactone), PLA (polyactic acid), PBAT (poly butylene adipate terephthalate) and mixtures thereof in any proportions.

According to one preferred embodiment, the biodegradable polyester (b), different from PHA, is PLA.

The biodegradable polyester (b), different from PHA, content is advantageously greater than 2%, preferably comprised between 2% and 75% by weight, preferably between 3% and 50% by weight, more preferably between 5% and 30%, more preferably still between 6% and 25% by weight relative to the total weight of the composition of the article.

The biodegradable polyester (b), different from PHA, is advantageously PLA and the PLA content is greater than 2%, preferably comprised between 2% and 75% by weight, preferably between 3% and 50% by weight, more preferably between 5% and 30%, more preferably still between 6% and 25% by weight relative to the total weight of the composition of the article. In another embodiment, the PLA content is equal to around 6.5%, 7.5%, 13%, 15%, 19.5%, 22.5% or 25% by weight relative to the total weight of the composition of the article.

The composition of the single-layer article according to the invention can optionally comprise in addition at least one complementary thermoplastic polymer (d), different from PHA (a) and the biodegradable polyester (b) defined above. These complementary thermoplastic polymers (d) are preferably biodegradable polyesters well-known to a person skilled in the art, and are in particular selected from PBAT (polybutylene adipate terephthalate), PBS (polybutylene succinate), PCL (polycaprolactone), PBSA (polybutylene succinate adipate), plasticised starch and mixtures thereof in any proportions. Thus, within the scope of the invention, the expression “complementary thermoplastic polymer” refers to additional biodegradable polyesters, different from PHA (a) and the biodegradable polyester (b) defined above. In other words, according to this embodiment, the article according to the invention comprises PHA and at least two biodegradable polyesters which distinct from PHA (a) and different from one another, the first of which is the biodegradable polyester (b) defined above, preferably PLA, and the second is at least one complementary thermoplastic polymer (d).

The content of complementary thermoplastic polymers (d), different from PHA (a) and the biodegradable polyester (b), is preferably comprised between 10% and 90%, between 20% and 80%, preferably between 30 and 75%, more preferably 40% and 70% by weight relative to the total weight of the composition of the article.

According to one embodiment, the complementary thermoplastic polymer (d) is selected from PBAT (polybutylene adipate terephthalate), PBS (poly butylene succinate), PCL (polycaprolactone) and PBSA (polybutylene succinate adipate). According to one specific embodiment, the complementary polymer (d) is PBAT.

According to another preferred embodiment, the composition of the article comprises a mixture comprising PHA, PBAT, the PBAT being the complementary thermoplastic polymer (d), PLA as biodegradable polymer (b), and enzymes capable of breaking down the PLA.

The composition of the article according to the invention comprises at least 0.001% enzymes capable of breaking down the biodegradable polyester, the percentage of enzymes being expressed by weight relative to the total weight of the composition of the article.

Enzymes capable of breaking down polyesters used within the scope of the present invention are well-known to a person skilled in the art. Reference is made in particular to enzymes capable of breaking down PLA so as to improve the biodegradability of the single-layer article according to the invention. Such enzymes and their method of incorporation into thermoplastic articles are known to a person skilled in the art, in particular described in the patent applications WO 2013/093355, WO 2016/198652, WO 2016/198650, WO 2016/146540 and WO 2016/062695. These enzymes are preferably selected from proteases and serine proteases. In one specific embodiment, the serine proteases are selected from Proteinase K from Tritirachium album, or PLA-degrading enzymes from Amycolatopsis sp., Actinomadura keratinilytica, Laceyella sacchari LP175. Thermus sp, or Bacillus licheniformis or reformulated commercial enzymes known to break down PLA such as Savinase®, Esperase®, Everlase® or any enzyme from the subtilisin family CAS 9014-01-1 or any functional variant.

A person skilled in the art is able to adapt the enzyme content depending on their objectives for breaking down the biodegradable polyester, and in particular the PLA of the single-layer article.

The enzyme content in the single-layer article will advantageously be at least 0.001%, or even at least 0.002%, more advantageously at least 0.05% by weight relative to the total weight of the composition of the article. These contents may go up to 10%, or even more. Although it is possible to formulate compositions comprising more than 10% of enzymes by weight, it is nevertheless rare to exceed such contents for the most common uses of plastic articles according to the invention. The enzyme content in the composition of the article is advantageously 0.01% to 7%, preferably 0.02 to 5% by weight relative to the total weight of the composition of the article.

The article may also comprise common additive used in the preparation of plastic articles, such as slip agents, plasticisers, nucleating agents, compatibilisers, manufacturing aids, UV stabilisers, anti-shock agents, mineral or plant-based fillers, etc.

The content of common additives in the single-layer article according to the invention is advantageously at least 0.2%, preferably 1 to 5%, more preferably 2 to 4%, advantageously around 2% by weight relative to the total weight of the composition of the article.

According to one preferred embodiment, the single-layer article according to the invention comprises one or more slip agents, such as oleamide, erucamide, stearamide, behenamide, oleyl palmitamide, stearyl erucamide, ethylene bis-oleamide, EBS or a mixture thereof. The slip agent is preferably oleamide.

The article may also comprise plasticisers. Plasticisers used in degradable plastic articles are well-known to a person skilled in the art. They are in particular selected from polyols and amides, lactic acid oligomers (LAOs) and citrate esters.

LAOs are plasticisers known to a person skilled in the art, in particular as bio-based materials. They are lactic acid oligomers with a molecular weight of less than 1500 g/mol. They are preferably esters of lactic acid oligomers, their carboxylic acid end being blocked by esterification with an alcohol, in particular a linear or branched C1-C10 alcohol, advantageously a C6-C10 alcohol, or a mixture thereof. Reference is made in particular to LAOs described in patent application EP 2 256 149 with their method of preparation, and LAOs marketed by the company Condensia Quimica under the brand name Glyplast®, in particular the Glyplast® OLA2 references, which has a molecular weight of 500 to 600 g/mol and Glyplast® OLA8 which has a molecular weight of 1000 to 1100 g/mol. According to one preferred embodiment of the invention, the LAOs have a molecular weight of at least 900 g/mol, preferably 1000 to 1400 g/mol, more preferably 1000 to 1100 g/mol.

Citrate esters are also plasticisers known to a person skilled in the art as bio-based materials. Reference is made in particular to triethyl citrate (TEC), triethyl acetyl citrate (TEAC), tributyl citrate (TBC), tributyl acetyl citrate (TBAC), preferably TBAC.

Compatibilisers can be used in the composition of the single-layer article according to the invention. Such PLA/polyester compatibilisers are well known to a person skilled in the art, in particular molecules with epoxy, acrylate, anhydride, oxazoline and lactam functions, which enable grafting reactions.

Compatibilisers include more specifically polyacrylates, terpolymers of ethylene, acrylic ester and glycidyl methacrylate (for example marketed under the brand name Lotader® by Arkema), PLA-PBAT-PLA triblock copolymers, maleic anhydride-grafted PLA (PLA-g-AM) or maleic anhydride-grafted PBAT (PBAT-g-AM).

According to one preferred embodiment of the invention, the compatibiliser is selected from polyacrylates, advantageously chosen from methacrylate derivatives, preferably the compatibiliser is poly(ethylene-co-methyl acrylate-co-glycidyl methacrylate). Such compatibilisers are well-known and described in particular by Dong & al. (International Journal of Molecular Sciences, 2013, 14, 20189-20203) and Ojijo & al. (Polymer 2015, 80, 1-17). One preferred compatibiliser is poly(ethylene-co-methyl acrylate-co-glycidyl methacrylate) marketed under the name JONCRYL® ADR-4468-by BASF.

Mineral and/or plant-based fillers can also be used in the composition of the single-layer article of the invention. Mineral fillers preferably used are calcium carbonate or talc.

Plant-based fillers preferably used are starch and wood fibres and flour.

The article according to the invention can also comprise barrier materials well-known to a person skilled in the art, and in particular EVOH (Poly(vinyl alcohol-co-ethylene), PGA (polyglycolic acid), cellulose and its derivatives, or polysaccharides and mixtures thereof in any proportions. These barrier materials are well-known to a person skilled in the art and can be used on their own or mixed with the aforementioned biodegradable polyester.

The PGA used as barrier material in the preparation of the article corresponds to polyglycolic acid, the melting temperature of which is 220° C. and the glass transition temperature of which is 40° C.

Cellulose includes more specifically microfibrillated cellulose (MFC) and cellulose acetate.

The MFC used as barrier material has a diameter comprised between 4 and 10006 nm and a density comprised between 0.51 and 1.57 g/cm3.

Polysaccharides used as barrier materials are also known to a person skilled in the art. Reference is made more specifically to galactomannans, pectin or soluble soy bean polysaccharides, marine extracts such as carrageenans and alginates, and microbial or animal polysaccharides such as gellans, dextrans, xanthans, xylans or chitosan, and mixtures thereof.

The proportion of bio-based material used to make the article according to the invention is advantageously greater than 30%, preferably greater than 40%.

According to one specific embodiment of the invention, the composition of the single-layer article comprises, by weight relative to the total weight of the composition of the article:
a) 10% to 35% PHA, preferably between 12.5 and 25%,
b) 20% to 50% PLA, preferably between 24 and 30%,
c) at least 0.001% enzymes capable of breaking down said PLA,
d) 42% to 60% PBAT, preferably between 49 and 56% and
e) optionally, 0.8% to 8% of a carrier polymer such as PCL or PBSA, preferably 4%.

According to one specific preferred embodiment of the invention, the composition of the single-layer article comprises, by weight relative to the total weight of the composition of the article:
a) 10% to 35% PHA, preferably between 12.5 and 25%,
b) 20% to 50% PLA, preferably between 24 and 30%,
c) at least 0.001% enzymes capable of breaking down said PLA,
d) 42% to 60% PBAT, preferably between 49 and 56% and
e) 0.8% to 8% PCL, preferably 4%.

According to another specific preferred embodiment of the invention, the composition of the single-layer article comprises, by weight relative to the total weight of the composition of the article:
a) 10% to 35% PHA, preferably between 12.5 and 25%,
b) 20% to 50% PLA, preferably between 24 and 30%,
c) at least 0.001% enzymes capable of breaking down said PLA,
d) 42% to 60% PBAT, preferably between 49 and 56% and
e) 0.8% to 8% PBSA, preferably 4%.

### Articles

According to the invention, the plastic article is a manufactured product, such as rigid or flexible packaging, agricultural films, pouches and bags, disposable items or similar articles. The article is preferably made of thermoplastic material. The plastic articles according to the invention may contain supplementary substances or additives, such as slip agents, plasticisers, and mineral or plant-based fillers.

The single-layer article according to the invention has a thickness greater than or equal to around 20 μm, to around 25 μm, to around 50 μm, to around 450 μm, to around 750 μm and less than or equal to 5000 μm.

According to the invention, the plastic article may be chosen from a flexible plastic article or a rigid plastic article.

According to one embodiment, the single-layer article according to the invention is a flexible article, in particular a plastic film.

Examples of plastic films include agricultural films, plastic bags or pouches, flexible packaging films, food films, seals, blister packs, mailing films, liner films, multipack films, industrial films, personal care films, nets, etc.

The flexible single-layer article according to the invention advantageously has a thickness ranging from 20 to 100 μm, preferably from 25 to 80 μm, more preferably from 30 to 60 μm, more preferably still of around 50 μm.

According to one embodiment, the single-layer article according to the invention is a rigid article.

Examples of rigid plastic articles include thin-walled packaging, packaging for food and beverages, boxes, trays, containers, food service items, electronic packages, make-up bags, outdoor gardening items such as pots, rigid packaging, containers, cups, plates, beverage capsules, tubs, cards, cotton wool pads, hose irrigation products, etc. Some rigid plastic articles can be produced by thermoforming plastic sheets with a thickness of 250 μm or more, these plastic sheets being produced by film casting or calendering.

According to the invention, the rigid plastic article has a thickness of less than 5000 μm, preferably less than 3000 μm, more preferably between 400 and 500 μm, more preferably still equal to around 450 μm. Alternatively, the thickness of the single-layer rigid article can be equal to around 750 μm.

The invention also relates to a sheet comprising a single-layer article according to the invention, preferably a rigid single-layer article as defined above.

The invention also relates to a film comprising a single-layer article according to the invention, preferably a flexible single-layer article as defined above.

The relative thickness of the article according to the invention may vary depending on the final properties required for the article, in particular in terms of water barrier property but also in terms of biodegradability.

The single-layer article of the invention may also be applied to a support of a different kind, such as a biodegradable film, a paper or other. For example, the single-layer article of the invention may be laminated onto a biodegradable film to enhance its water barrier properties.

### Preparation of the Article

Another object of the invention relates to a method for preparing a single-layer article according to the invention. The articles are prepared by mixing the various components and then shaping the article to the required shape, whether flexible or rigid.

According to one specific embodiment of the single-layer article according to the invention, the method comprises the following steps:
a) providing enzymes having polyester degrading activity in a form suitable to allow homogeneous dispersion of said enzymes in the single-layer article, said form being selected from
   a liquid composition comprising the enzymes having polyester degrading activity, water and optionally a polysaccharide carrier, or
   a masterbatch comprising the enzymes having polyester degrading activity and a carrier polymer having a melting temperature below 140° C. and/or a glass transition temperature below 70° C., and
b) mixing the enzymes having a polyester degrading activity provided in step (a) with the PHA, the biodegradable polyester, different from PHA, and possibly at least one complementary thermoplastic polymer different from PHA and the biodegradable polyester, and
c) shaping the article.

A person skilled in the art will know how to prepare the single-layer article according to the invention by any common technique, such as extrusion blow-moulding, extrusion calendering, injection-moulding, in particular by extrusion blow-moulding. The article is preferably prepared by extrusion, a person skilled in the art being able to determine the conditions for carrying out the method, preferably in the proportions defined above. Extrusion is advantageously carried out at a temperature below 200° C.

According to a first embodiment, step a) comprises providing a liquid composition comprising the enzymes having polyester degrading activity, water and optionally a polysaccharide carrier. Such liquid compositions and their use in preparing enzyme-containing polymer mixtures are described in particular in applications WO 2019/043145 and WO 2019/043134.

The content of enzyme composition used to prepare the single-layer article will depend in particular on the enzyme content in the enzyme composition and the desired enzyme content of the article. According to one specific embodiment, 1 to 10% by weight of enzyme composition is used relative to the total weight of the composition of the article.

According to a second embodiment, the enzymes are supplied by a masterbatch.

The masterbatch is a concentrated premix of enzymes and a carrier polymer. The carrier polymer comprised in the masterbatch is different from PHA (a), the biodegradable polyester (b) and the potential complementary thermoplastic polymer (c) defined above.

The carrier polymer is a polymer having a melting temperature below 140° C. and/or a glass transition temperature below 70° C.

Polymers having a melting temperature below 140° C. and/or a glass transition temperature below 70° C. are well-known to a person skilled in the art. They are in particular PCL (polycaprolactone), PBSA (polybutylene succinate adipate), PBAT (poly butylene adipate terephthalate), PHA (polyhydroxyalkanoate), PLA (polylactic acid), or copolymers thereof. It may also be a natural polymer such as starch or a polymer that would be considered universal, i.e. compatible with a wide range of polymers such as an EVA (ethylene-vinyl acetate)-type copolymer. The carrier polymer advantageously has a melting temperature below 120° C. and/or a glass transition temperature below 30° C.

The carrier polymer is preferably selected from PCL, PBSA, PBAT, PHA, PLA and mixtures thereof in any proportions, in particular PCL or more preferably still PBSA.

The masterbatch preferably also comprises a polysaccharide.

Polysaccharides are in particular selected from starch derivatives such as amylose, amylopectin, maltodextrins, glucose syrup, dextrins, and cyclodextrins, natural gums such as gum arabic, tragacanth gum, guar gum, locust bean gum, karaya gum, mesquite gum, galactomannans, pectin or soluble soybean polysaccharides, marine extracts such as carrageenans and alginates, and microbial or animal polysaccharides such as gellans, dextrans, xanthans or chitosan, and mixtures thereof. A preferred polysaccharide is arabic gum.

In this case, the composition of the single-layer article according to the invention also comprises a polysaccharide.

A preferred masterbatch composition comprises from 50 to 95% of a low melting point polymer, in particular PCL (polycaprolactone) or PBSA (polybutylene succinate adipate), preferably from 70 to 90% by weight relative to the total weight of the masterbatch, from 0.001 to 10% of enzymes, preferably from 1 to 6% by weight relative to the total weight of the masterbatch, and from 1 to 30% of gum arabic, preferably from 10 to 25% by weight relative to the total weight of the masterbatch.

Such masterbatches and their preparation methods are described in particular in patent applications WO 2019/043134, WO 2021/148665 or in French patent application no. 2107809 filed on 20 Jul. 2021.

According to another embodiment, the article is prepared by shaping a premix or “compound” consisting of the composition of the article defined above.

The premix is prepared according to the method described above, with granulation using conventional methods for shaping step c).

The invention therefore also relates to a premix in the form of granules comprising:
a) from 3% to less than 60% PHA (polyhydroxyalkanoate)
b) a biodegradable polyester, different from PHA (a),
c) at least 0.001% enzymes capable of breaking down said polyester,
d) optionally, at least one complementary thermoplastic polymer, different from PHA (a) and the biodegradable polyester (b),
   the percentages being expressed in weight relative to the total weight of the premix.

All of the alternative embodiments described above for the single-layer article of the invention apply mutatis mutandis to the premix.

### Use of the Articles

The invention also relates to various uses of the single-layer articles obtained according to the invention, in particular single-layer flexible or rigid articles.

The single-layer article according to the invention may be used for all common uses of thermoplastic articles, and in particular for the preparation of packaging or bags, or for mulching or mailing. It may also be used for the preparation of disposable tableware (plates, cups), packaging (tubs, blister packs), seals or beverage capsules.

The various embodiments, alternatives, preferences and advantages described above for each of the objects of the invention apply to all of the objects of the invention and can be considered separately or in combination.

The invention is illustrated by the following examples provided in a non-limiting manner.

### Examples

### I. Preparing a Mix of Carrier Polymer and Enzyme

The carrier polymer and enzyme mix is prepared from polycaprolactone (PCL) granules and the enzyme in liquid form as described in the application WO 2019/043134.

The carrier polymer and enzyme mix was produced using a CLEXTRAL EV25HT twin-screw extruder comprising 11 zones for which the temperature is independently controlled and regulated. The PCL is introduced into zone 1 at 16 kg/h and the enzyme solution into zone 5 at 4 kg/h using a peristaltic pump. The zones are heated according to table [1]. 20% of the enzyme solution is added to the PCL (% by weight relative to the total weight).

TABLE 1
Temperature profile (° C.) used
for the carrier polymer and enzyme mix

| Zone | Z1 | Z2 | Z3 | Z4 | Z5 | Z6 | Z7 | Z8 | Z9 | Z10 | Z11 |
|---|---|---|---|---|---|---|---|---|---|---|---|
| Temperature | 40 | 65 | 75 | 75 | 60 | 60 | 60 | 60 | 60 | 60 | 60 |

### II. Commercial Products

In these examples, PHBV marketed under the reference ENMAT™ Y1000P by Helian Polymers, PHBH marketed under the reference Green PLlanet™ X128 by KANEKA, PLA/PBAT mixtures marketed under the references Ecovio® F2331, Ecovio® F2223, Ecovio® F2332 and a slip agent under the reference Ecoflex® SL-05 marketed by BASF, PLA marketed under the reference Luminy® LX175 by Total Corbion.

### III. Producing the Single-Layer Films

To prepare single-layer films, a LabTech LF-250 blown film extrusion line with an LBE20-10 30/C extruder is used, with a diameter of 20 mm and screw ratio of L/D=30. The screw speed was around 50 rpm.

The settings and temperatures for a film containing PHBV are detailed in tables and [3].

TABLE 2
Extruder temperature and process parameters for a film containing PHBV
Temperature profile

| Heating zone | Z1 | Z2 | Z3 | Z4 | Z5 H | Z6 B | Material |
|---|---|---|---|---|---|---|---|
| Setpoint | 175 | 175 | 175 | 175 | 160 | 160 | 178 |

TABLE 3
Extruder process parameters for a film containing PHBV

| | |
|---|---|
| Pressure (bar) | 163 |
| Screw speed (RPM) | 50 |
| Torque (Nm) | 71 |
| High draw speed (m/min) | 4.2 |
| Low draw speed (m/min) | 4.5 |
| Ventilation speed (RPM) | 500 |

The settings and temperatures for a film containing PHBV are detailed in tables [4] and [5].

TABLE 4
Extruder temperature parameters for a film containing PHBH
Temperature profile

| Heating zone | Z1 | Z2 | Z3 | Z4 | Z5 H | Z6 B | Material |
|---|---|---|---|---|---|---|---|
| Setpoint | 155 | 160 | 160 | 160 | 165 | 160 | 168 |

TABLE 5
Extruder process parameters for a film containing PHBH
Process parameters

| | |
|---|---|
| Pressure (bar) | 141 |
| Screw speed (RPM) | 55 |
| Torque (Nm) | 70 |
| High draw speed (m/min) | 2.1 |
| Low draw speed (m/min) | 2.4 |
| Ventilation speed (RPM) | 800 |

### IV. Analysis Method

The water barrier properties of the films was evaluated with a permeability test according to the following protocol: films measuring 15×17 cm were sealed on three ends to form a bag which was then filled with 20.0 g of water. The bag was then sealed on its last side to form an airtight pouch and laid flat for 7 days. The mass of the bag was determined every day for a week to obtain an average water loss in g/d.

The biodegradability of the films was evaluated using a depolymerisation test carried out according to the following protocol: 100 mg of each sample was introduced into a plastic vial containing 50 mL of buffer solution at pH 9.5. Depolymerisation was initiated by incubating each sample at 45° C. in an incubator shaken at 150 RPM. A 1 mL aliquot of the buffer solution was taken regularly and filtered using a 0.22 μm filter syringe for analysis by high-performance liquid chromatography (HPLC) using an Aminex HPX-87H column to measure the release of lactic acid (LA) and its dimer. The chromatography system used is an Ultimate 3000 UHPLC system (Thermo Fisher Scientific, Inc. Waltham, MA, USA) comprising a pump, an automatic sampler, a column temperature-controlled at 50° C. and a 220 nm UV detector. The eluent is 5 mM H₂SO₄. The injection is 20 μL of sample. Lactic acid is measured using standard curves prepared from commercial lactic acid.

The hydrolysis of the plastic films is calculated from the lactic acid and lactic acid dimer released. The percentage of depolymerisation is calculated in relation to the percentage of PLA in the sample.

### VII. Composition of the Films and Results

The single-layer films were prepared using the polymer carrier and enzyme mix prepared in I, PHA of the PHBV or PHBH type, PLA, and a mixture of PLA/PBAT.

The first series of single-layer films was produced with a thickness of 25 μm.

The compositions of these different films are listed in table [6] below.

TABLE 6
Composition of the different single-layer films

| | | | | Ecovio ® | |
|---|---|---|---|---|---|
| | | | | F2223 | Carrier |
| | | Green | | (mixture | polymer |
| | ENMATTM | PlanetTM | | 30% | and enzyme |
| Film | Y1000P | X128 | PLA | PLA/70% | mix prepared |
| 25 μm | (PHBV) | (PHBH) | LX175 | PBAT) | in I |
| 1 C | 0% | 0% | 0% | 100% | 0% |
| 2 C | 15% | 0% | 0% | 85% | 0% |
| 3 | 15% | 0% | 0% | 75% | 10% |
| 4C | 25% | 0% | 0% | 75% | 0% |
| 5 | 25% | 0% | 0% | 65% | 10% |
| 6 C | 0% | 0% | 95% | 0% | 5% |
| 7C | 0% | 25% | 0% | 75% | 0% |
| 8 | 0% | 25% | 0% | 65% | 10% |
| 9C | 60% | 0% | 0% | 30% | 10% |

Examples 1C, 2C, 4C, 6C, 7C and 9C are comparative examples.

The formulation in example 9C (with a PHA content of over 60%, in particular PHBV) was difficult to extrude to obtain films and the material was very sticky, destabilising the bubble and preventing the tubes from opening.

The properties of the films prepared above were measured according to the protocols defined above.

The results of these measurements are provided in table 7 below.

TABLE 7
Results of the measurements

| Film | Biodegradability | Water barrier properties |
|---|---|---|
| 25 μm | (% PLA depolymerised) | (water loss g/d) |
| 1 C | 0 | 1.7 |
| 2 C | 0 | 0.60 |
| 3 | 80 | 0.88 |
| 4C | 0 | 0.50 |
| 5 | 95 | 0.62 |
| 6 C | 90 | 1.9 |
| 7C | 0 | 1.09 |
| 8 | 95 | 0.92 |
| 9C | — | — |

This shows that the polymer mixture according to the invention results in improved water barrier properties compared to a film without PHA. PHBV improves the barrier properties in PLA mixtures more significantly than PHBH.

The second series of films was produced with a thickness of 35 μm.

The compositions of these different films are listed in table [8] below.

TABLE 8
Composition of the different single-layer films

| | ENMATTM | | | Carrier polymer |
|---|---|---|---|---|
| Film | Y1000P | Green PlanetTM | PLA | and enzyme mix |
| 35 μm | (PHBV) | X128 (PHBH) | LX175 | prepared in I |
| 10 | 0% | 20% | 75% | 5% |
| 11 | 0% | 40% | 55% | 5% |
| 12C | 0% | 95% | 0% | 5% |
| 13 | 0% | 0% | 95% | 5% |

Example 12C is a comparative example.

The properties of the films prepared above were measured according to the protocols defined above.

The results of these measurements are provided in table 9 below.

TABLE 9
Results of the measurements

| Film | Biodegradability | Water barrier properties |
|---|---|---|
| 35 μm | (% PLA depolymerised) | (water loss g/d) |
| 10 | 56 | 0.53 |
| 11 | 70 | 0.38 |
| 12C | — | 0.81 |
| 13 | 70 | 1.6 |

The formulation in comparative example 12C (with a PHBH content of 95% and PLA content of 0%) has the worst barrier properties in the whole series of examples.

This shows that the polymer mixture according to the invention results in improved water barrier properties compared to a pure PHA or PLA film. PHBV improves the barrier properties in PLA mixtures more significantly than PHBH.

1. Biodegradable single-layer article, the composition of which comprises:
a) from 3% to less than 60% PHA (polyhydroxyalkanoate)
b) a biodegradable polyester, different from PHA (a),
c) at least 0.001% enzymes capable of breaking down said biodegradable polyester, and
d) optionally at least one complementary thermoplastic polymer, different from PHA (a) and the biodegradable polyester (b),
the percentages being given in weight relative to the total weight of the composition of the article.

2. Article according to claim 1, characterised in that the PHA (a) content is comprised between 5% and 50%, preferably between 10% and 30%, more preferably still between 12% and 27%.

3. Article according to claim 1 or 2, characterised in that the biodegradable polyester (b) is PLA (polyactic acid).

4. Article according to one of claims 1 to 3, characterised in that the biodegradable polyester (b) content is greater than 2%, preferably comprised between 2% and 75% by weight, preferably between 3% and 50% by weight, more preferably between 5% and 30%, more preferably still between 6% and 25%.

5. Article according to one of claims 1 to 4, characterised in that the complementary thermoplastic polymers (d) are selected from PBAT (polybutylene adipate terephthalate), PBS (polybutylene succinate), PCL (polycaprolactone), PBSA (polybutylene succinate adipate), plasticised starch and mixtures thereof in any proportions.

6. Article according to one of claims 1 to 5, characterised in that the complementary thermoplastic polymer (d) content is comprised between 10% and 90%, preferably between 30 and 75%, more preferably 40% and 70%.

7. Article according to one of claims 1 to 6, characterised in that it comprises a polysaccharide.

8. Article according to one of claims 1 to 7, characterised in that it comprises, by weight relative to the total weight of the composition of the article:
10% to 35% PHA, preferably between 12.5 and 25%
20% to 50% PLA, preferably between 24 and 30%
42% to 60% PBAT, preferably between 49% and 56%
0.8% to 8% PCL or PBSA, preferably 4%
at least 0.001% enzymes capable of breaking down said PLA.

9. Article according to one of claims 1 to 8, characterised in that the PHA (a) is selected from PHBV (poly-3-hydroxybutyrate-co-3-hydroxyvalerate), PHBH (poly-3-hydroxy butyrate-co-3-hydroxyhexanoate), P3HP (poly 3-hydroxypropionate), P3HB (poly 3-hydroxybutyrate), P4HB (poly 4-hydroxybutyrate), P4HV (poly 4-hydroxyvalerate), P5HV (poly 5-hydroxyvalerate), PHB3HP (poly 3-hydroxybutyrate-co-3-hydroxypropionate), P3HB4HB (poly 3-hydroxy butyrate-co-4-hydroxybutyrate), PHB4HV (poly 3-hydroxy butyrate-co-4-hydroxyvalerate), PHB3HV (poly 3-hydroxybutyrate-co-3-hydroxyvalerate), PHB3HH (poly 3-hydroxy butyrate-co-3-hydroxyhexanoate), PHB5HV (poly 3-hydroxybutyrate-co-5-hydroxyvalerate) and mixtures thereof in any proportions.

10. Article according to one of claims 1 to 9, characterised in that it comprises, in addition, at least one barrier material selected from EVOH (Poly(vinyl alcohol-co-ethylene)), PGA (polyglycolic acid), cellulose or polysaccharides and mixtures thereof in any proportions.

11. Article according to one of claims 1 to 10, characterised in that it also comprises at least one additive selected from slip agents, plasticisers, nucleating agents, compatibilisers, manufacturing aids, UV stabilisers, anti-shock agents, mineral or plant-based fillers and mixtures thereof.

12. Article according to one of claims 1 to 11, characterised in that it has a thickness greater than or equal to around 20 μm, to around 25 μm, to around 50 μm, to around 450 μm, to around 750 μm and less than or equal to 5000 μm.

13. Article according to one of claims 1 to 12, characterised in that it is a sheet.

14. Article according to claim 13, characterised in that it has a thickness between 400 and 500 μm, preferably equal to around 450 μm.

15. Article according to one of claims 1 to 12, characterised in that it is a film.

16. Article according to claim 15, characterised in that it has a thickness ranging from 20 to 100 μm, preferably from 25 to 80 μm, more preferably from 30 to 60 μm, more preferably still of around 50 μm.

17. Method for preparing an article according to one of claims 1 to 16, characterised in that it comprises the following steps:
a) providing enzymes having polyester degrading activity in a form suitable to allow homogeneous dispersion of said enzymes in the single-layer article, said form being selected from
a liquid composition comprising the enzymes having polyester degrading activity, water and optionally a polysaccharide carrier, or
a masterbatch comprising the enzymes having polyester degrading activity and a carrier polymer having a melting temperature below 140° C. and/or a glass transition temperature below 70° C., and
b) mixing the enzymes having a polyester degrading activity provided in step (a) with the PHA (a), the biodegradable polyester (b) and possibly at least one complementary thermoplastic polymer (d), and
c) shaping the article.

18. Use of an article according to one of claims 1 to 16 for preparing a package, seal, cup, plate, beverage capsule, tray or blister pack.
